(19) United States
(12) Patent Application Publication
(10) Pub. No.: US 2021/0030458 A1
(43) Pub. Date: Feb. 4, 2021

(54) ESOPHAGEAL PROBES AND METHODS

(71) Applicant: E-SAFE Medical, Inc., Houston, TX (US)

(72) Inventors: Ashok Gowda, Bellaire, TX (US); Charles Houssiere, Houston, TX (US); Joshua G. Corso, Houston, TX (US)

(21) Appl. No.: 16/964,195

(22) PCT Filed: Jan. 23, 2019
(86) PCT No.: PCT/US19/14832

§ 371 Date: Jul. 22, 2020

Related U.S. Application Data
(60) Provisional application No. 62/620,814, filed on Jan. 23, 2018.

(51) Int. Cl. A61B 18/04; A61M 31/00; A61B 90/00

(52) U.S. Cl. CPC A61B 18/04; A61B 2018/00815; A61B 90/04; A61M 31/005

(57) ABSTRACT

The invention relates to devices and methods for affecting an internal body tube, such as the esophagus, particularly to affecting the internal body tube by inserting a device into the internal body tube and more particularly to affecting the internal body tube by at least partially sealing off a section and moving the walls of the internal body tube, and/or applying cooling to the internal body tube. This invention further relates to methods of using such devices to move portions of an internal body tube away from an area undergoing a treatment or therapy, such as to minimize damage to the internal body tube, and/or providing cooling/temperature monitoring.

### CROSS-REFERENCE TO RELATED APPLICATIONS

This Patent Cooperation Treaty international application claims the benefit and priority of U.S. provisional patent application Ser. No. 62/620,814, filed Jan. 23, 2018, entitled “ESOPHAGEAL PROBES AND METHODS”, the entire contents of which is hereby incorporated by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### FIELD OF THE INVENTION

The invention relates to devices and methods for affecting an internal body tube, such as the esophagus, particularly to affecting the internal body tube by inserting a device into the internal body tube and more particularly to affecting the internal body tube by at least partially sealing off a section and moving the walls of the internal body tube, and/or applying cooling to the internal body tube. This invention further relates to methods of using such devices to move portions of an internal body tube away from an area undergoing a treatment or therapy, such as to minimize damage to the internal body tube, and/or providing cooling/temperature monitoring.

### BACKGROUND OF THE INVENTION

Atrial fibrillation ablation involves delivery of radiofrequency energy to the left atrium. RF energy leads to thermal damage and ablation of tissue. This is the primary means by which it creates is therapeutic effects. Because of its proximity to the esophagus, ablation of the left atrium (especially the posterior wall or in the regions around the pulmonary veins) can lead to delivery of RF energy and thermal damage at the left atrium. Because of its close proximity to the esophagus the energy delivery and secondary lesions may extend to esophageal tissue. This is a potential and likely mechanism of atrioesophageal fistula (AEF), a rare but lethal complication of the procedure. Approximately 75% of patients who have AEF will die from the condition.

There has been a steady increase in the body of literature suggesting that the most effective means of protecting a patient from AEF is by avoiding ablation in the regions close to the esophagus. A variety of techniques have attempted to define the relative position of the esophagus to the left atrium during the course of ablation. These include placement of a thermal probe in the form of a radioopaque or radiolucent catheter. The probe delineates the overall outline of the esophagus as it traverses along the posterior aspect of the left atrium. It will also alert the operator whether or not RF application is leading to increased local (intraluminal) esophageal temperatures. Such temperature increments have been theorized to serve as a trigger for fistula formation. Given the relative infrequency of these complications it is difficult to assess what effect, if any, these interventions have on the development of AEF.

What is clear to every practitioner of ablation (cardiac electrophysiologists) is that ablation in regions close to the esophagus is to be avoided at all costs. Unfortunately these areas are often the major triggers of atrial fibrillation in the first place. Thus avoiding ablation in the regions adjacent to the esophagus may in fact lead to lower success rates of the ablation procedure and lower rate of cure of atrial fibrillation. It is thus desirable to be able to displace the esophagus selectively so that in the course of ablation it can be removed from the proximity of the ablation catheter. Towards this end nasogastric tubes with deflection catheters have been suggested to be effective. There are currently technologies that use deflection of an endoscopic or nasogastric tube to achieve this. These technologies, however, require use of a more expensive deflectable catheter and manipulation of the catheter so that the esophagus is in fact mobilized effectively. This can be quite cumbersome and may necessitate use of additional personnel. Given the large numbers of such procedures the economic impact of using mechanical deflection of the esophagus using endoscopes and trained personnel will likely be prohibitive. Furthermore the entire esophageal profile as it courses over the thermal probe is usually not known unless contrast is used.

### SUMMARY OF THE INVENTION

The invention relates to devices and methods for affecting an internal body tube, such as the esophagus, particularly to affecting the internal body tube by inserting a device into the internal body tube and more particularly to affecting the internal body tube by at least partially sealing off a section, moving the walls of the internal body tube, and/or applying cooling to the internal body tube. This invention further relates to methods of using such devices to move portions of an internal body tube away from an area undergoing a treatment or therapy and/or providing cooling/temperature monitoring, such as to minimize damage to the internal body tube.

In general, a device for affecting an internal body tube may be utilized on a variety of body structures, such as, for example, the esophagus. The device may generally include a tube which is inserted into an internal body tube proximal to an area to be affected by the device.

In one aspect of the invention, the device may include a tube with at least one fenestration or other port through which cooling fluid may be delivered to the area surrounding the tube and at least one other fenestration or other port through which to withdraw the cooling fluid for removal and/or recirculation. The fenestrations or ports may be connected to external sources of cooling fluid and/or circulation lines, such as through internal lumens formed into the tube or with internal tubes or conduits within the tube, such that cooling fluid may be continuously delivered to lower or modulate the temperature surrounding the tube.

In some embodiments, the tube may include a plurality of fenestrations or ports which may be utilized to apply suction or other means to different locations along the tube or, for example, to equalize the applied suction or other means along a larger area of the tube than with a single fenestration or port. The fenestrations or ports may be connected to an external source of suction or other means, such as through internal lumens formed into the tube or with internal tubes or conduits carried within the tube.

In another aspect of the invention, the device may further include at least one sealing feature to isolate a portion of internal body tube to be affected by the device. In general, the sealing feature may form at least a partial seal against the wall of the internal body tube. Sealing features may include, for example, deformable or compressible formations about the tube of the device. In some embodiments, the sealing feature may include a balloon or other feature that may be selectively expanded to contact the wall of the internal body tube or deflated/contracted to draw away from the wall, such as to aid in moving the device in the internal body tube without additional contact or friction with the wall. The balloon or other feature may also generally be connected to a fluid or gas source or withdrawing device, such as a suction line or pump, and the connection may generally be carried by lumen(s) or conduit(s)/tube(s) within the tube and connected to an external source or device.

In some embodiments, the device may include at least two sealing features along the length of the tube, such as, for example, that the fenestrations or ports (or groupings of them) lie between the two sealing features. The two sealing features may then, for example, seal off a particular length of the internal body tube at either end of the particular length to be affected by the device. In other embodiments, three or more sealing features may be utilized to, for example, create multiple separate particular lengths to be affected separately. Where balloons or other selectively expandable/contractible feature is utilized, they may be individually controlled or controlled in groups. In still other embodiments, the length of tube between two sealing features may be adjustable, such as to provide variable lengths for cooling and/or to accommodate different sized internal body tubes.

In a further aspect of the invention, the device may further include sensors or other features for aiding in locating the device within the internal body tube and/or for detecting a desired area of the internal body tube to be affected by the device. In some embodiments, desired areas of the internal body tube may be determined by detected conditions in the area, such as temperature, pressure, electrical resistance/conductivity, and/or any other appropriate condition or combination thereof. For example, temperature may be detected to determine where an internal body tube may need to be acted on, such as, for example, to aid in protecting it from damage from a temperature increase in the vicinity, and/or for modulating the application of cooling fluid to control the temperature in the vicinity. For further example, pressure or other indicators of contact of the wall of the internal body tube with the tube of the device may be utilized to determine the degree the device is affecting the internal body tube. This may be desirable, for example, to control the degree of suction or drawing force to prevent damage or excess deformation of the internal body tube. Sensors or other features which gather data or information from the vicinity of the device may output to an external display such that the information may be monitored during a procedure. In some embodiments, the external display may be movable relative to a handpiece or other controller for the device, such as through a wired or wireless connection, such that the external display may be placed in a convenient location for viewing by a user of the device, such as in a preferred line of sight.

In another aspect of the invention, the device may be used to apply suction or other drawing or deflecting action to the inside of an internal body tube to cause the internal body tube to contract, deform, deflect or decrease in, for example, size, diameter or caliber. In some embodiments, the device may be utilized to draw the esophagus walls inward, such as to move the esophagus or portion thereof away from another structure, such as away from portions of the heart. This may be desirable as treatments or therapies on the heart, such as, for example, cardiac ablation procedures, may cause damage to adjacent structures, such as the esophagus. In some embodiments, deflection may be accomplished by altering the shape of the device when inserted in an internal body tube, such as be inducing curvature or bending, such as with a deflecting feature in the device or by altering the shape of a portion of the device.

Use of the device on the esophagus in areas adjacent to an area undergoing treatment may aid in spacing the esophagus away from the treatment area to protect it from or at least decrease incidental damage. In general, the application of suction to draw in and deflect the esophagus away from the treatment area may result in an atraumatic deflection without application of more traumatic forms of external force to cause the deflection. In some embodiments, the device may also employ sensors, such as discussed above, to monitor the location where temperatures are increased during a treatment to control the location and degree of suction or other drawing force being applied to the esophagus. The sensors may also, for example, act in a feedback control mechanism on the suction or other drawing force, or, for further example, in controlling or influencing the degree of temperature change being induced in the adjacent therapy. Temperature sensors may be utilized, such as, for example, thermocouples, thermistors, non-contact temperature sensors (e.g. infrared (IR) or other radiation sensing thermometers) and/or any other appropriate form of temperature sensing device or component.

In a further aspect of the invention, the device may be utilized to deliver a fluid or substance to an internal body tube, such as a contrast agent or other detectable substance. In some exemplary embodiments, the device may deliver a quantity of a contrast agent or other detectable substance to coat the inside of the internal body tube, after which the excess may be suctioned or otherwise removed to leave a residual detectable layer. This may be desirable to provide a detectable residue which may be utilized to monitor the position, shape and/or size of the internal body tube during a procedure. In some embodiments, the contrast agent or detectable substance may be selected to at least partial adsorb into the internal body tube such that it remains in place during the course of procedure and/or otherwise resist immediate removal, such as during the circulation of cooling fluid during a procedure.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIGS. 1 and 1b illustrate the external features of a device for affecting an internal body tube with sealing features;

FIG. 1a illustrates the external features of a device for affecting an internal body tube without sealing features;

FIG. 1c illustrates a device for affecting an internal body tube with an adjustable length portion;

FIGS. 2, 2a and 2b illustrate a device coupled with a handpiece and external display;

FIG. 2c illustrates a device with a gravity-driven fluid delivery system;

FIGS. 3 and 3a illustrate a device with a deformable curvature; and

FIGS. 4, 4a and 4b illustrate usage of a device to apply a detectable substance in an internal body tube.

### DETAILED DESCRIPTION OF THE INVENTION

The detailed description set forth below is intended as a description of the presently exemplified methods, devices and systems provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and systems similar or equivalent to those described herein can be used in the practice or testing of the invention, the exemplified methods, devices and systems are now described.

The invention relates to devices and methods for affecting an internal body tube, such as the esophagus, particularly to affecting the internal body tube by inserting a device into the internal body tube and more particularly to affecting the internal body tube by at least partially sealing off a section, moving the walls of the internal body tube, and/or applying cooling to the internal body tube. This invention further relates to methods of using such devices to move portions of an internal body tube away from an area undergoing a treatment or therapy and/or providing cooling/temperature monitoring, such as to minimize damage to the internal body tube.

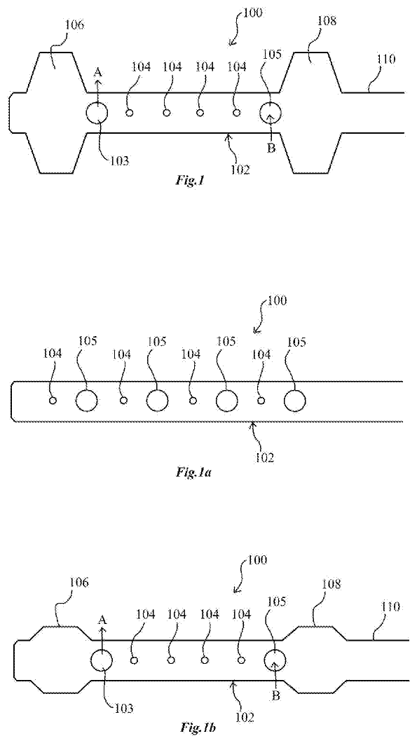

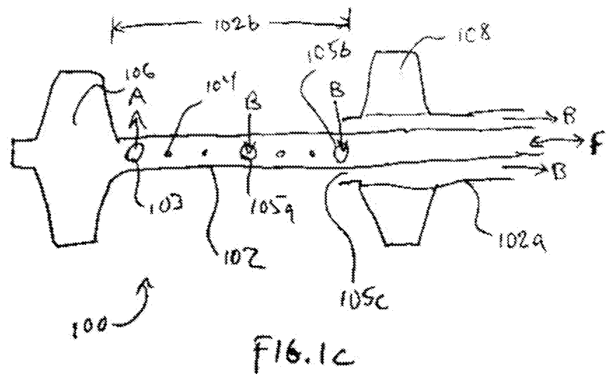

In general, a device for affecting an internal body tube may be utilized on a variety of body structures, such as, for example, the esophagus. The device may generally include a tube which is inserted into an internal body tube proximal to an area to be affected by the device. FIGS. 1, la and lb illustrate embodiments of a device 100 for affecting an internal body tube including a tube portion 102 which is inserted into an internal body tube. The materials used for the inserted portions of the device 100, such as the portions on and disposed adjacent to the tube portion 102, may generally be selected to be non-toxic, biocompatible or otherwise safe for internal use in the body.

In one aspect of the invention, the device may include a tube with at least one fenestration or other port through which suction or other means of drawing fluid or gas may be applied to the area surrounding the tube. In some embodiments, a single fenestration or port may be utilized to apply suction or other means, such as to remove fluid from the vicinity, as illustrated with uptake B from fenestration 105 in FIGS. 1 and 1b. In some embodiments, the tube may include a plurality of fenestrations or ports which may be utilized to apply suction or other means to different locations along the tube or, for example, to equalize the applied suction or other means along a larger area of the tube than with a single fenestration or port, as illustrated with fenestrations 105 in tube portion 102 of the device 100 in FIG. 1a. The fenestrations or ports may be connected to an external source of suction or other means, such as through internal lumens formed into the tube or with internal tubes or conduits carried within the tube, as illustrated with the conduit tube 110 that connects to the fenestrations 103 through the proximal end of the tube portion 102.

The fenestrations or ports may take any appropriate form or number along the tube portion 102. In general, it may be desirable that the size and shape are appropriate to apply suction without clogging or causing incidental damage by drawing in significant amounts of tissue from the wall of the internal body tube. For example, when used for the esophagus, some embodiments may utilize circular or ellipsoidal fenestrations of less than about 0.5 cm diameter. For further example, the length of the tube portion 102 with the fenestrations may be on the order of about 8 or 8.5 cm, such as to, for example, accommodate an appropriate length of esophagus.

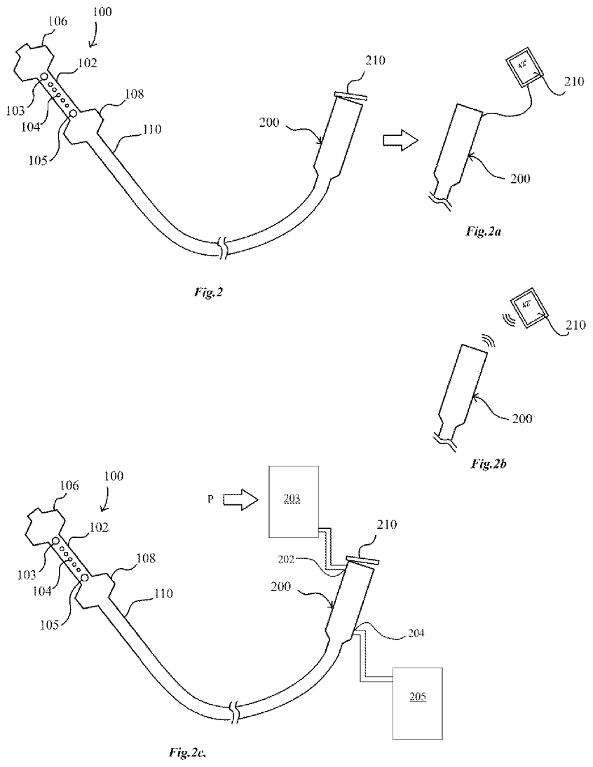

In one aspect of the invention, the device may include a tube with at least one fenestration or other port through which cooling fluid may be delivered to the area surrounding the tube and at least one other fenestration or other port through which to withdraw the cooling fluid for removal and/or recirculation. The fenestrations or ports may be connected to external sources of cooling fluid and/or circulation lines, such as through internal lumens formed into the tube or with internal tubes or conduits within the tube, such that cooling fluid may be continuously delivered to lower or modulate the temperature surrounding the tube. FIGS. 1 and 1b illustrate the delivery of fluid A from a fenestration 103 into the vicinity of the tube portion 102 which is then removed by uptake B at fenestration 105. In some embodiments, the fluid A may be supplied by an external source to fenestration 103 from a location higher than the uptake point at fenestration 105 or higher than the outflow collection connected to fenestration 105. This may enable the use of gravity to drive the flow of fluid A from fenestration 103 and its exit through fenestration 105. This may be desirable to enable flow of the fluid A without the use of a pump or other mechanism to provide flow. In some examples, an IV bag or similar reservoir may be utilized to supply the fluid A. The IV bag or similar reservoir may also be pressurized in a controlled manner such that it may, for example, be utilized to alter the flow rate. For example, a cuff (e.g. an inflatable cuff or the like) or other external source of pressure may be applied to the IV bag or similar reservoir to alter the pressure inside. FIG. 2c illustrates an example of a device 100 with a fluid source 203 connected to a connection point 202 which is connected to fenestration 103 and a collection reservoir 205 connected to a connection point 204 which is connected to fenestration 105. As illustrated, the collection reservoir 205 is lower than the fluid source 203, which may enable gravity to power the flow of a fluid from the fluid source 203 through fenestration 103 with uptake at fenestration 105 and flow into the collection reservoir 205. Pressure in the fluid source 203 may be altered by applying a given pressure P on the fluid source 203, such as with a cuff (e.g. inflatable cuff or the like) or other pressure source (e.g. a pump, plunger, piston, etc.).

In another aspect of the invention, the device may further include at least one sealing feature to isolate a portion of internal body tube to be affected by the device. FIGS. 1 and 1b illustrate a device 100 with at least two sealing features, shown with sealing balloons 106, 108, with sealing balloon 106 at the distal end of the tube portion 102 and the sealing balloon 108 at the proximal end of the tube portion 102. In general, the sealing feature may form at least a partial seal against the wall of the internal body tube. Sealing features may include, for example, deformable or compressible formations about the tube of the device. In some embodiments, the sealing feature may include a balloon or other feature that may be selectively expanded to contact the wall of the internal body tube, as shown with the inflated state in FIG. 1, or deflated/contracted to draw away from the wall as shown in FIG. 1b, such as to aid in moving the device in the internal body tube without additional contact or friction with the wall. The balloon or other feature may also generally be connected to a fluid or gas source or withdrawing device, such as a suction line or pump, and the connection may generally be carried by lumen(s) or conduit(s)/tube(s) within the tube and connected to an external source or device, as illustrated with the handpiece 200 connected through the conduit tube 110 to the sealing balloons 106, 108, respectively, as shown in FIG. 2. The handpiece 200 may include or be connected to, for example, a syringe, pump or other inflation/deflation device. The handpiece 200 may further include locks or other mechanisms to maintain the level of inflation or deflation in the sealing balloons 106, 108, such as with locking valves, which may, for example, prevent backflow of inflation gas or fluid from the sealing balloons 106, 108, such as due to pressure on them in situ. Other selective sealing features may also be utilized, such as mechanically deploying struts, expanding stents, flattening/widening seals and/or any other appropriate feature or combination thereof.

In some embodiments, the sealing balloons 106, 108 may be connected to source(s) of a given fixed volume, such as fixed volume syringes or the like, such that the overall amount of gas or fluid to inflate the sealing balloons 106, 108 may be limited and/or controlled. This may be desirable to prevent over-inflation, which may apply too much pressure and/or otherwise damage the internal body tube.

In some embodiments, the device may include at least two sealing features along the length of the tube, such as, for example, that the fenestrations or ports (or groupings of them) lie between the two sealing features. The two sealing features may then, for example, seal off a particular length of the internal body tube at either end of the particular length to be affected by the device. In other embodiments, three or more sealing features may be utilized to, for example, create multiple separate particular lengths to be affected separately. Where balloons or other selectively expandable/contractible feature is utilized, they may be individually controlled or controlled in groups.

In still other embodiments, the length of tube between two sealing features may be adjustable, such as to provide variable lengths for cooling and/or to accommodate different sized internal body tubes. FIG. 1c illustrates an embodiment of a device 100 which includes an inner tube portion 102 which is translatable along direction F within an outer sheath 102 such that translation along direction F may allow the exposed length 102b of inner tube portion 102 to be adjusted, such as to accommodate different working lengths and/or to expose/cover fenestrations or sensors, as shown with fenestrations 103, 105a, 105b and sensors 104. In some embodiments, fluid uptake B may also be accomplished with the annulus 105c between the inner tube portion 102 and the outer sheath 102.

In a further aspect of the invention, the device may further include sensors or other features for aiding in locating the device within the internal body tube and/or for detecting a desired area of the internal body tube to be affected by the device. FIGS. 1 and 1a illustrate a plurality of sensors 104 disposed along the length of the tube portion 102. In some embodiments, desired areas of the internal body tube may be determined by detected conditions in the area, such as temperature, pressure, electrical resistance/conductivity, and/or any other appropriate condition or combination thereof. For example, temperature may be detected to determine where an internal body tube may need to be acted on, such as, for example, to aid in protecting it from damage from a temperature increase in the vicinity and/or for providing feedback/control to modulate the cooling fluid being applied, such as by modulation of temperature, flow rate, etc. This may utilize, for example, thermocouples, thermistors, non-contact temperature sensors (e.g. infrared (IR) or other radiation sensing thermometers) or other forms of temperature sensors as the sensors 104. The sensors 104 may also include, for example, location tracking features such as MRI or CT fiducials, Hall Effect magnetic sensors or other appropriate location tracking features. For further example, pressure or other indicators of contact of the wall of the internal body tube with the tube of the device may be utilized to determine the degree the device is affecting the internal body tube. This may be desirable, for example, to control the degree of suction or drawing force to prevent damage or excess deformation of the internal body tube. Sensors 104, for example, could detect the pressure of the wall of the internal body tube acting on the outside of the tube portion 102. The sealing features may also feature detection features for determining the level or state of deployment of the seal, such as a pressure sensor for use with inflating balloons.

As illustrated in FIG. 2, the sensors 104 may be connected to at least one external device, such as to handpiece 200 via the conduit tube 110 from a connection to the sensors 104 in the tube portion 102. For example, the handpiece 200 may be utilized to monitor the temperature or other sensor feedback, and/or provide feedback control signals. Sensors or other features which gather data or information from the vicinity of the device may output to an external display 210 such that the information may be monitored during a procedure. In some embodiments, the external display 210 may be movable relative to a handpiece or other controller for the device, such as through a wired connection as illustrated in FIG. 2a, or wireless connection as illustrated in FIG. 2b, such that the external display may be placed in a convenient location for viewing by a user of the device, such as in a preferred line of sight. For example, the external display 210 may include an attachment or mounting feature(s), such as adhesives, magnetic elements, clips, stands and/or other attachment/mounting mechanisms to accommodate placement in a preferred location.

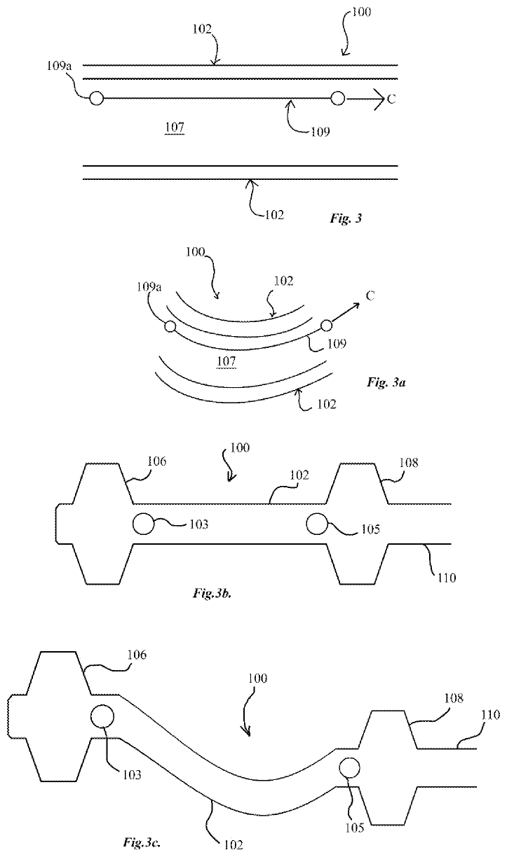

In another aspect of the invention, the device may be used to apply suction or other drawing or deflecting action to the inside of an internal body tube to cause the internal body tube to contract, deform, deflect or decrease in, for example, size, diameter or caliber. In some embodiments, the device may be utilized to draw the esophagus walls inward, such as to move the esophagus or portion thereof away from another structure, such as away from portions of the heart, such as with device 100 in FIG. 1a which utilizes fenestrations 105 (shown without sealing features such as balloons). This may be desirable as treatments or therapies on the heart, such as, for example, cardiac ablation procedures, may cause damage to adjacent structures, such as the esophagus. In some embodiments, deflection may be accomplished by altering the shape of the device when inserted in an internal body tube, such as be inducing curvature or bending. FIGS. 3 and 3a illustrate usage of a deflecting element, such as pull-wire 109 as illustrated, to alter the curvature or shape of the tube portion 102 of the device 100. As illustrated, for example, the pull-wire 109 may be anchored on a portion 109a attached to the tube portion 102, such as in a lumen 107, such that when the pull-wire 109 is pulled C, the tube portion 102 along the pull-wire 109 may contract, resulting in the generation of curvature or deformation, as shown in FIG. 3a.

In other embodiments, the tube portion 102 of the device 100 may be utilized to alter the shape of the device when inserted in an internal body tube. FIGS. 3b and 3c illustrate an example of the device 100 where at least a portion of the tube portion 102 may be inflatable to change its shape from a resting state, as illustrated in FIG. 3b, to a deflecting state with an altered shape, as illustrated in FIG. 3c. For example, at least a portion of the tube portion 102 may be a balloon which has little form or structure when deflated, but assumes a more rigid form of a particular shape when inflated. This may be desirable as the force from the deflection of a balloon may be, for example and without being bound to any particular theory, distributed over a larger area on the inflated balloon and/or provide a gentler deflection such as to reduce any trauma to an internal body tube during the deflection. In some embodiments, the inflatable portion may be independent from sealing features 106, 108, if present. In other embodiments, the inflatable portion may be integral to or connected with one or both sealing features 106, 108 such that their inflation and/or deflation may be connected. Fenestrations, such as the fenestrations 103, 105 as illustrated, may be integrated into the form of the tube portion 102 such that they are not connected to the inflatable portions, so that they may operate independently. Sensors or other devices (e.g. temperature sensors), may also be included on the surface of the inflatable portion or in direct contact with the wall of the inflatable portion such that they may measure in the vicinity of the tube portion 102.

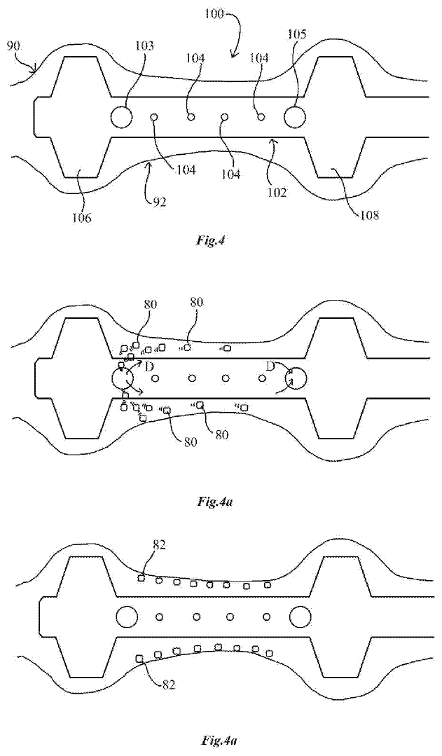

In a further aspect of the invention, the device may be utilized to deliver a fluid or substance to an internal body tube, such as a contrast agent or other detectable substance. In some exemplary embodiments, the device may deliver a quantity of a contrast agent or other detectable substance to coat the inside of the internal body tube, after which the excess may be suctioned or otherwise removed to leave a residual detectable layer. FIGS. 4, 4a and 4b illustrate use of the device 100 for applying a detectable substance to the interior of an internal body tube 90, such as a region to be monitored 92 between the sealing features 106, 108. As illustrated, a detectable substance, such as contrast agent 80, may be released in a flow D from fenestration 103 to fill the region 92. The contrast agent 80 may be allowed to incubate for a time such that it may form a layer or be absorbed into a portion of the region 92. The excess contrast agent 80 may then be taken up through fenestration 105, as illustrated in FIG. 4a. This may be desirable to provide a detectable residue, as shown with remaining residue 82 in FIG. 4b, which may be utilized to monitor the position, shape and/or size of the internal body tube during a procedure. In some embodiments, the contrast agent or detectable substance may be selected to at least partial adsorb into the internal body tube such that it remains in place during the course of procedure and/or otherwise resist immediate removal, such as during the circulation of cooling fluid during a procedure. Also, as illustrated in FIGS. 4, 4a and 4b, the sealing features 106, 108 may be utilized to block off the section of the internal body tube 90 such as to, for example, substantially or completely block flow of the contrast agent 80 into other parts of the internal body tube 90, such as by abutting against and sealing against the walls of the internal body tube 90. This may be desirable, for example and without being bound by any particular theory, in preventing contrast agent 80 from flowing into undesirable areas, such as into the stomach when the internal body tube 90 is the esophagus. Further, the sealing by sealing features 106, 108 may, without being bound to any particular theory, allow larger concentrations of contrast agent 80 to be utilized than would be desirable otherwise, such as if the contrast agent 80 was able to flow into the stomach or other undesirable areas. Also, preventing flow into the stomach or other undesirable areas may allow for usage of a smaller amount of contrast agent 80, as without the prevention of flow, some of the contrast agent 80 may flow out of the desired area and thus may not be useful. The contrast agent 80 may also be substantially removed, such as by suction or other uptake into the device 100, and/or re-administered, for example multiple times, to provide contrast agent 80 when desired or needed without reaching any maximum amount of contrast agent 80 to be administered since excess is removed and/or not allowed to flow into the stomach or other undesirable areas.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

1. A device for affecting an internal body tube comprising:
a tube portion having at least a first internal lumen connecting to at least one exit fenestration disposed on an outer surface toward a distal end and a second internal lumen connecting to at least one uptake fenestration disposed on said outer surface toward a proximal end;
a connecting conduit coupled to the proximal end of said tube portion, said connecting conduit housing a suction line connected to said second internal lumen and interfacing with a suction source and a delivery line connected to said first internal lumen and interfacing with a fluid source;
a first sealing feature disposed at said proximal end of said tube portion; and
a second sealing feature disposed at said distal end of said tube portion;
wherein said tube portion is adapted for insertion into an internal body tube of a mammalian subject and said first and second sealing features form reversible seals against the walls of said internal body tube.

2. A method for affecting an internal body tube comprising:
inserting a device into a portion of an internal body tube of a mammalian subject, said device comprising:
a tube portion having at least a first internal lumen connecting to at least one exit fenestration disposed on an outer surface toward a distal end and a second internal lumen connecting to at least one uptake fenestration disposed on an outer surface toward a proximal end;
a connecting conduit coupled to the proximal end of said tube portion, said connecting conduit housing a suction line connected to said second internal lumen and interfacing with a suction source and a delivery line connected to said first internal lumen and interfacing with a fluid source;
a first sealing feature disposed at said proximal end of said tube portion; and
a second sealing feature disposed at said distal end of said tube portion;
forming a sealed area of said portion by sealing said first and second sealing features against a wall of said internal body tube; and
applying a cooling fluid exiting from said exit fenestration and uptake of said cooling fluid at said uptake fenestration;
wherein said cooling fluid cools said sealed area of said internal body tube.

3. A method for affecting an internal body tube comprising:
inserting a device into a portion of an internal body tube of a mammalian subject, said device comprising:
a tube portion having at least a first internal lumen connecting to at least one exit fenestration disposed on an outer surface toward a distal end and a second internal lumen connecting to at least one uptake fenestration disposed on an outer surface toward a proximal end;
a connecting conduit coupled to the proximal end of said tube portion, said connecting conduit housing a suction line connected to said second internal lumen and interfacing with a suction source and a delivery line connected to said first internal lumen and interfacing with a fluid source;
a first sealing feature disposed at said proximal end of said tube portion; and
a second sealing feature disposed at said distal end of said tube portion;
forming a sealed area of said portion by sealing said first and second sealing features against a wall of said internal body tube; and
applying a contrast agent exiting from said exit fenestration and uptake of said contrast agent at said uptake fenestration;
wherein said contrast agent forms a detectable residue on said sealed area of said internal body tube.

4. The device of claim 1, wherein said first and second sealing features each comprise a sealing balloon attached to at least one inflation/deflation device through said connecting conduit with a locking mechanism and a fixed volume of inflation fluid or gas.

5. The device of claim 1, wherein said second internal lumen connects to a plurality of fenestrations disposed at intervals on said outer surface of said tube portion.

6. The device of claim 1, further comprising at least one sensor disposed on said tube portion and having an output, said at least one sensor being selected from the group consisting of a temperature sensor, a pressure sensor, an electrical conductivity/resistance sensor, a position sensor, an Mill fiducial and a CT fiducial.

7. The method of claim 2, further comprising at least one temperature sensor disposed on said tube portion and having an output that controls the power output of a thermal ablation device.

8. The device of claim 1, wherein said tube portion has an adjustable length.

9. The device of claim 1, further comprising at least one external display showing output from at least one sensor selected from the group consisting of a temperature sensor, a pressure sensor, an electrical conductivity/resistance sensor, a position sensor, an MM fiducial and a CT fiducial.

10. The device of claim 1, further comprising at least one external display coupled to a handpiece in communication with said device, said at least one external display being detachable and movable relative to said handpiece showing output from at least one sensor selected from the group consisting of a temperature sensor, a pressure sensor, an electrical conductivity/resistance sensor, a position sensor, an MM fiducial and a CT fiducial.

11. The device of claim 1, further comprising at least one additional tube portion at said distal end of said tube portion with an additional sealing feature disposed between each tube portion.

12. The device of claim 1, wherein suction at each of said at least one fenestrations is separately controlled or controlled in groupings.

13. The method of claim 2, wherein said forming said sealed area and applying said cooling fluid are performed after detecting a temperature change in said internal body tube.

14. The device claim 1, further comprising a pull-wire for deforming said tube portion.

15. The device of claim 1, wherein said internal body tube comprises an esophagus.

16. The device of claim 1, further comprising an inflatable portion of said tube portion for deforming said tube portion.

17. The device of claim 1, wherein gravity drives flow from said fluid.

18. The device claim 1, further comprising a shaped balloon portion of said tube portion adapted for deforming said tube portion when said shaped balloon portion is inflated.

19. The device of claim 1, further comprising a temperature sensor disposed on said device selected from the group consisting of a thermocouple, a thermistor, and an infrared temperature sensor.
